(19) United States
(12) Patent Application Publication
(10) Pub. No.: US 2022/0110885 A1
(43) Pub. Date: Apr. 14, 2022

(54) TRANSDERMAL THERAPEUTICS SYSTEM WITH BARRIER LAYER

(71) Applicant: LTS Lohmann Therapie-Systeme AG, Andemach (DE)

(72) Inventor: Dieter PAULUKAT, Kruft (DE)

(73) Assignee: LTS Lohmann Therapie-Systeme AG, Andernach (DE)

(21) Appl. No.: 17/278,844

(22) PCT Filed: Sep. 19, 2019
(86) PCT No.: PCT/EP2019/075132

§ 371 Date: Mar. 23, 2021

(30) Foreign Application Priority Data
Sep. 24, 2018 (DE) 10 2018 216 244.1

(51) Int. Cl. A61K 9/70

(52) U.S. Cl. CPC A61K 9/7084

(57) ABSTRACT

The invention relates to a transdermal therapeutic system that includes a) a backing layer which faces away from the skin, is impermeable to the active substance, and is equipped with an adhesive layer for fixing onto the skin, b) an active substance reservoir which contains at least one active substance, c) a barrier layer which faces the skin, adjoins the active substance reservoir, is impermeable to the active substance and has at least one opening, and d) a removable protective layer, in which the backing layer which is equipped with the adhesive layer projects beyond the barrier layer on all sides.

In common transdermal therapeutic systems, the active substance is released onto the skin relatively rapidly, since the entire surface area of the active-substance-containing matrix lies against the skin. Active substance release is usually effected in a membrane-controlled or diffusion- controlled manner. An additional form of control of active substance release is not however provided by such transdermal therapeutic systems.

Various approaches for the controlled release of active substances are described in the literature. U.S. Pat. No. 4,927,687 discloses a transdermal system for the controlled release of steroids that comprises a backing layer, a protective layer, an active substance reservoir, an adhesive layer, and a membrane with macropores. The active substance reservoir consists of a viscous base material and microcapsules that contain the appropriate steroid and through which the rate of release is controlled.

EP 0 387 693 A2 discloses a transdermal system with stepwise active substance release that is used for local or systemic dermal administration of active substances in human or veterinary medicine or in cosmetics. The transdermal therapeutic system described in EP 0 387 693 A2 has a backing layer, an active substance reservoir, a control membrane with openings that is preferably introduced into the active substance reservoir thereby dividing it, a pressure- sensitive-adhesive layer, and a protective layer. The control membrane, the membrane surface area of which is smaller than the release surface area of the system, ensures that release takes place according to zero-order kinetics when the active substance is present in the reservoir in a supersaturated concentration and according to first-order kinetics when the active substance is present in the reservoir below this concentration. Such “stepwise” release is desirable in some special cases when there is no need for active substance release to take place at a constant rate and thus for even, constant release.

Another approach is described in WO 99/07349. This document discloses a transdermal therapeutic system comprising an active-substance-containing matrix having a skin-facing pressure-sensitive-adhesive active layer, wherein the active layer of the matrix is at the time of application covered, in part of the area of its active-substance-releasing surface facing the skin, with an active substance-impermeable barrier layer that is formed as a circular surface. This system has the disadvantage that active substance release is limited to the area of skin that lies under the outer region of the transdermal therapeutic system and is not covered by the barrier layer. Moreover, with this system it is possible for active substance to escape via the lateral edges of the active substance reservoirs.

The object of the present invention is to provide a transdermal therapeutic system that allows a low dosage of an active substance and at the same time ensures that the transdermal therapeutic system can be worn for a long time without becoming detached from the skin prematurely.

This object is achieved by the transdermal therapeutic system according to the invention.

Transdermal therapeutic systems (TTS) are systems for the controlled administration of active pharmaceutical ingredients through the skin. They have long since been used to treat various diseases, physical and mental disorders, complaints, and ailments. Transdermal therapeutic systems are layered products in the form of patches comprising a backing layer impermeable to the active substance, at least one active-substance-containing reservoir layer or matrix layer, and a removable protective layer that is peeled off the TTS before use.

To attach a transdermal therapeutic system to the skin and to ensure the controlled administration of the active substance, the TTS is provided with a pressure-sensitive- adhesive layer. This pressure-sensitive-adhesive layer may be identical to the active-substance-containing matrix layer or to the skin-facing active-substance-containing layer, but may also be additionally present when the (skin-facing) active-substance-containing layer or the optionally present membrane is not pressure-sensitive-adhesive.

The backing layer of a TTS needs to be impermeable to the active substance present in the TTS in order to prevent undesired leakage of the active substance from the side of the TTS that is facing away from the skin. Metal foils, special plastic films, and composite laminates of these materials are in particular used for this purpose. The most common are composite laminates made of aluminum and plastic materials such as polyethylene terephthalate. The advantage of these composite laminates is that aluminum foils can be produced inexpensively and are impermeable to almost all active pharmaceutical ingredients. Aluminum foils are also opaque, which, with light-sensitive active substances in particular, offers the advantage of reliable protection from light.

The present invention relates to a transdermal therapeutic system comprising
a) a backing layer that is facing away from the skin and impermeable to the active substance and that is furnished with a pressure-sensitive-adhesive layer for attachment to the skin,
b) an active substance reservoir comprising at least one active substance,
c) a barrier layer that is facing the skin and that adjoins the active substance reservoir, is impermeable to the active substance, and has at least one opening,
d) a removable protective layer,
wherein the backing layer that is furnished with the pressure-sensitive-adhesive layer protrudes beyond the barrier layer on all sides.

According to the invention, the barrier layer protrudes beyond the active substance reservoir on all sides. This ensures that the active substance from the active substance reservoir is able to diffuse into the skin only via the at least one opening in the barrier layer, active substance release thus being controlled via the at least one opening in the barrier layer.

The barrier layer must be of a chosen thickness that achieves an adequate barrier effect, that is to say it is not possible for any active substance to diffuse through the barrier layer. According to the invention, the barrier layer has a thickness from 5 to 20 μm, preferably 8 to 17 μm, more preferably from 10 to 15 μm.

The at least one opening in the barrier layer is circular, oval or polygonal in shape, wherein it is preferable that the at least one opening in the barrier layer is circular in shape.

The size and number of the at least one opening depends on how high active substance release is to be. The lower the desired release of active substance, the smaller the at least one opening. According to the invention, the at least one opening has an area from 0.07 to 176.72 mm², preferably from 0.19 to 78.54 mm², more preferably from 0.78 to 19.64 mm².

When the at least one opening is circular in shape, according to the invention the opening has a diameter of 0.3-15 mm, preferably of 0.5 to 10 mm, more preferably of 1 to 5 mm.

The number of the at least one opening is limited by the area of the barrier layer and the area of the active substance reservoir. In addition to the desired amount of active substance that is to be released, the number of the at least one opening has an influence on the size of the at least one opening. In general, the more openings there are, the smaller the area of the at least one opening, or the fewer openings there are, the larger the area thereof.

The choice of the suitable number and size of the at least one opening in the barrier layer allows the release of a specific amount of active substance over a relatively long period of time.

The active substance-impermeable backing layer and the barrier layer may consist of the same materials or of different materials. Suitable materials for the active substance-impermeable backing layer and the barrier layer are especially polyesters characterized by particular strength, for example polyethylene terephthalate and polybutylene terephthalate, but also almost any other skin-friendly plastics such as polyvinyl chloride, polyurethane, polyvinylidene chloride, ethylene-vinyl acetate copolymers, polyvinyl acetate, vinyl acetate-vinyl chloride copolymers, nylon, polyethylene, polypropylene, polyurethanes, polyamide, cellulose derivatives, and many others besides. Preference is given to polyethylene terephthalate, plasticized vinyl acetate-vinyl chloride copolymers, nylon, ethylene-vinyl acetate copolymers, plasticized polyvinyl chloride, polyurethane, polyvinylidene chloride, polypropylene, polyethylene, and polyamide. In individual cases, the backing layer may be provided with an additional overlay, for example by vapor deposition with metals, in particular aluminum.

The same materials may in principle be used for the removable protective layer as for the backing layer, provided they have been rendered removable through a suitable surface treatment such as siliconization. Other removable protective layers may however also be used, for example paper treated with polytetrafluoroethylene or ®Cellophane (cellulose hydrate).

Suitable base polymers for pressure-sensitive-adhesive layers are described for example in Tan, Pfister, PSTT vol. 2, No. 2, February 1999, pages 60-69. Examples of suitable base polymers are polyacrylates, poly(meth)acrylates, polyacrylic acid, cellulose derivatives, in particular methyl- and ethylcelluloses, polyisobutylene, ethylene-vinyl acetate copolymers, natural and synthetic rubbers such as styrene-diene copolymers, styrene-butadiene block copolymers, styrene-isoprene block copolymers, acrylonitrile-butadiene rubber, butyl rubber or neoprene rubber, and also hot-melt adhesives. Silicone-based adhesives are also suitable as pressure-sensitive adhesives. Suitable mixtures of the recited polymers or hybrid adhesives composed of acrylate monomers and silicone monomers may also be advantageously used.

The active substance reservoir of the transdermal therapeutic systems according to the invention may comprise various auxiliaries or additives, for example from the group of solubilizers, solvents, plasticizers, permeation improvers, pH regulators, antioxidants, and preservatives.

The active substance may be present in the active substance reservoir of the transdermal therapeutic system in combination with a solubilizer; it is also possible to use a mixture of solubilizers.

Examples of preferred solubilizers are polyhydric alcohols such as propane-1,2-diol, the various butanediols, glycerol, polyethylene glycol 400, tetrahydrofurfuryl alcohol, diethylene glycol monoethyl ether, diethyl toluamide, and monoisopropylidene glycerol. Particular preference is given to using propane-1,2-diol. Some of the solubilizers mentioned, including propane-1,2-diol, can additionally also have a permeation-promoting effect.

It has been found to be advantageous when the proportion of the solubilizer(s) is between 1% and 50% by weight, preferably between 5% and 35% by weight, based on the entire transdermal therapeutic system in the finished state after production.

The transdermal therapeutic system according to the invention may be produced both in the form of matrix systems and in the form of bag reservoir systems or membrane systems. The term “matrix systems” includes not only those systems in which the active substance is dissolved or dispersed in a layered synthetic resin or plastic matrix and is released therefrom, but also those in which the active substance is adsorbed on fiber material such as cotton woven or nonwoven fabric. This fiber material may be embedded in a plastic matrix or synthetic resin matrix.

A large number of polymers, resins, and additives may in principle be used to produce the active substance reservoir, provided the substances that come into contact with the skin are skin-friendly and as long as the formulation produced therewith is capable of releasing the active substance onto the skin.

The active substance reservoir is preferably formed as an active-substance-containing matrix, wherein said matrix is a synthetic resin matrix or plastic matrix that comprises, as base polymer(s), one or more polymers preferably selected from the group comprising polyacrylates, poly(meth)acrylates, polyacrylic acid, cellulose derivatives, isobutylene, ethylene-vinyl acetate, natural and synthetic rubbers such as styrene-diene copolymers, styrene-butadiene block copolymers, isoprene block copolymers, acrylonitrile-butadiene rubber, butyl rubber or neoprene rubber, and also hot-melt adhesives.

The active substance reservoir of the transdermal therapeutic system according to the invention may have a single-layer, double-layer or multilayer structure, i.e. the active substance reservoir may be formed as an active-substance- containing matrix, wherein the active substance reservoir consists of one, two or several matrix layers. The various matrix layers may differ in their composition or in the concentration of the constituents contained therein. For example, the various matrix layers may have a different polymer composition or consist of different pressure- sensitive adhesives.

The active substance may be present in the active substance reservoir in liquid or solid form. According to the present invention, it is preferable that the active substance reservoir is formed as an active-substance-containing matrix in which the active substance is present adsorbed on a fiber material, preferably on cotton woven or nonwoven fabric, wherein said fiber material is preferably embedded in a plastic matrix or synthetic resin matrix.

The backing layer furnished with the pressure-sensitive- adhesive layer projects beyond the barrier layer on all sides, thereby ensuing good adhesion of the transdermal therapeutic system to the skin. This pressure-sensitive- adhesive layer is completely sufficient for attachment of the transdermal therapeutic system on the skin. The active substance reservoir may however additionally be furnished with a pressure-sensitive-adhesive layer or may, on the skin- facing side at least, itself be formed as a pressure- sensitive-adhesive layer. The active substance reservoir is preferably formed as a pressure-sensitive-adhesive layer.

When the active substance reservoir is formed as a pressure- sensitive-adhesive layer, the active substance reservoir, or at least the layer of the matrix of the active substance reservoir facing the skin when the system is in use, comprises a pressure-sensitive-adhesive polymer or a combination of pressure-sensitive-adhesive polymers. For the purposes of the present description, “pressure-sensitive-adhesive polymers” are understood as meaning those polymers that are contained in pressure-sensitive adhesive formulations and that are suitable for use on the skin.

The pressure-sensitive-adhesive polymer is preferably selected from the group of polymers comprising, preferably consisting of, polyacrylates, polymethacrylates, polydimethylsiloxanes, polyvinyl acetates, polyisobutylenes, styrene-isoprene-styrene block copolymers, styrene- butadiene-styrene block copolymers, polyterpenes, ethylene- vinyl acetate copolymers, rubbers, and synthetic rubbers.

The proportion of the pressure-sensitive-adhesive polymer(s) is preferably from 5% to 90% by weight based on the active substance reservoir or on the pressure-sensitive-adhesive matrix layer(s).

The pressure-sensitive-adhesive polymer(s) of the matrix are preferably present in a crosslinked state. The crosslinking of the pressure-sensitive-adhesive polymers may be effected in ways known to those skilled in the art, for example by using chemical crosslinkers, for example aluminum acetylacetonate or titanium acetylacetonate in the case of polyacrylates, or by irradiation.

In addition or as an alternative to the active substance reservoir, the barrier layer may likewise be furnished with a pressure-sensitive-adhesive layer. This pressure- sensitive-adhesive layer likewise comprises a pressure- sensitive-adhesive polymer, wherein the pressure-sensitive- adhesive polymer is preferably selected from the group of polymers comprising, preferably consisting of, polyacrylates, polymethacrylates, polydimethylsiloxanes, polyvinyl acetates, polyisobutylenes, styrene-isoprene- styrene block copolymers, styrene-butadiene-styrene block copolymers, polyterpenes, ethylene-vinyl acetate copolymers, rubbers, and synthetic rubbers.

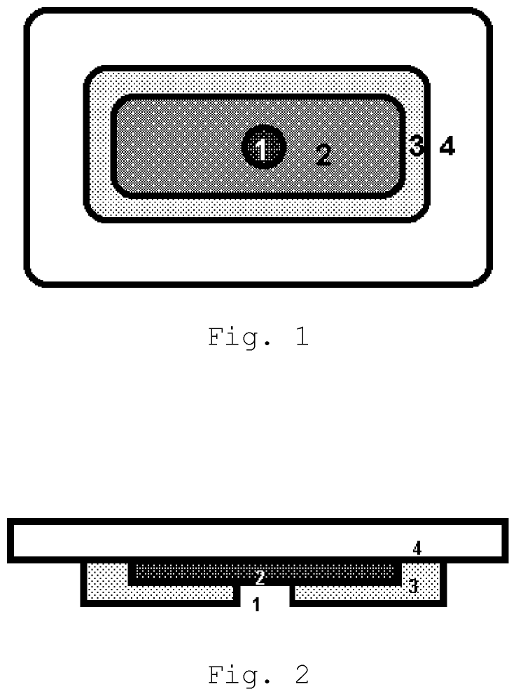

FIG. 1 shows a schematic representation of a TTS according to the invention.

FIG. 2 shows a lateral view of a TTS according to the invention. FIGS. 1 and 2 show a backing layer (4) impermeable to the active substance, an active substance reservoir (2) comprising at least one active substance, a skin-facing barrier layer (3), and an opening (1).

1. A transdermal therapeutic system comprising
a) a backing layer that is facing away from skin and is impermeable to active substance(s), said backing layer comprising a pressure-sensitive- adhesive layer for attachment to the skin,
b) an active substance reservoir comprising at least one active substance,
c) a skin-facing barrier layer that adjoins the active substance reservoir, is impermeable to the active substance, and has at least one opening,
d) a removable protective layer,
wherein the backing layer that is furnished with the pressure-sensitive-adhesive layer protrudes beyond the barrier layer on all sides and
wherein the barrier layer protrudes beyond the active substance reservoir on all sides.

2. The transdermal therapeutic system as claimed in claim 1, wherein the barrier layer has a thickness from 5 to 20 μm.

3. The transdermal therapeutic system as claimed in claim 1, wherein the at least one opening has an area from 0.07 to 176.72 mm².

4. The transdermal therapeutic system as claimed in claim 1, wherein the at least one opening in the barrier layer is circular, oval or polygonal in shape.

5. The transdermal therapeutic system as claimed in claim 1, wherein the active substance reservoir is a pressure-sensitive-adhesive layer.

6. The transdermal therapeutic system as claimed in claim 1, wherein the barrier layer comprises a pressure-sensitive-adhesive layer.

7. The transdermal therapeutic system as claimed in claim 1, wherein the barrier layer comprises a material selected from the group consisting of polyethylene terephthalate, plasticized vinyl acetate-vinyl chloride copolymers, nylon, ethylene-vinyl acetate copolymers, plasticized polyvinyl chloride, polyurethane, polyvinylidene chloride, polypropylene, polyethylene and polyamide.

8. The transdermal therapeutic system as claimed in one of claim 1, wherein the pressure-sensitive-adhesive layer comprises, as base polymers, polymers selected from the group consisting of polyacrylates, poly(meth)acrylates, polyacrylic acid, cellulose derivatives, polyisobutylene, ethylene-vinyl acetate copolymers, natural rubbers, synthetic rubbers, hot-melt silicone-based adhesives and pressure-sensitive silicone-based adhesives.

9. The transdermal therapeutic system as claimed in claim 1, wherein the active substance reservoir comprises at least one auxiliary or additive selected from the group consisting of solvents, solubilizers, plasticizers, permeation improvers, pH regulators, antioxidants, and preservatives.

10. The transdermal therapeutic system as claimed in claim 1, wherein the active substance reservoir comprises one or more solubilizers, and the concentration of the solubilizer(s) is between 1% and 50% by weight, based on the entire system.

11. The transdermal therapeutic system as claimed in claim 1, wherein the active substance reservoir is an active-substance-containing matrix, and the active substance reservoir consists of one, two or several matrix layers.

12. The transdermal therapeutic system as claimed in claim 1, wherein the active substance reservoir is an active-substance-containing matrix, and said matrix is a synthetic resin matrix or plastic matrix that comprises, as base polymer(s), one or more polymers selected from the group consisting of polyacrylates, poly(meth)acrylates, polyacrylic acid, cellulose derivatives, isobutylene, ethylene-vinyl acetate, natural rubbers, synthetic rubbers, and hot-melt adhesives.

13. The transdermal therapeutic system as claimed in claim 1, wherein the active substance reservoir is an active-substance-containing matrix in which the active substance is present adsorbed on a fiber material.

14. The transdermal therapeutic system as claimed in claim 1, wherein the active substance reservoir comprises a pressure-sensitive-adhesive polymer or a combination of pressure-sensitive-adhesive polymers, wherein the pressure-sensitive-adhesive polymer is selected from the group consisting of polyacrylates, polymethacrylates, polydimethylsiloxanes, polyvinyl acetates, polyisobutylenes, styrene-isoprene-styrene block copolymers, styrene-butadiene-styrene block copolymers, polyterpenes, ethylene-vinyl acetate copolymers, rubbers, and synthetic rubbers.

15. The transdermal therapeutic system as claimed in claim 2, wherein the barrier layer has a thickness from 8 to 17 μm.

16. The transdermal therapeutic system as claimed in claim 2, wherein the barrier layer has a thickness from 10 to 15 μm.

17. The transdermal therapeutic system as claimed in claim 3, wherein the at least one opening has an area from 0.19 to 78.54 mm².

18. The transdermal therapeutic system as claimed in claim 3, wherein the at least one opening has an area from 0.78 to 19.64 mm².

19. The transdermal therapeutic system as claimed in claim 8, wherein the synthetic rubbers are styrene-diene copolymers, styrene-butadiene block copolymers, styrene- isoprene block copolymers, acrylonitrile-butadiene rubber, butyl rubber or neoprene rubber.

20. The transdermal therapeutic system as claimed in claim 10 , wherein the solubilizers are polyhydric alcohols and the concentration of the solubilizer(s) is between 5% and 35% by weight, based on the entire system.

21. The transdermal therapeutic system as claimed in claim 10 , wherein the solubilizers are selected from the group consisting of propane-1,2-diol, butanediols, glycerol, polyethylene glycol 400, tetrahydrofurfuryl alcohol, diethylene glycol monoethyl ether, diethyl toluamide, and monoisopropylidene glycerol.

22. The transdermal therapeutic system as claimed in claim 12, wherein the synthetic rubbers are styrene-diene copolymers, styrene-butadiene block copolymers, isoprene block copolymers, acrylonitrile-butadiene rubber, butyl rubber or neoprene rubber.

23. The transdermal therapeutic system as claimed in claim 13, wherein the fiber material is a woven or nonwoven fabric comprised of cotton and said fiber material is embedded in a plastic matrix or synthetic resin matrix.
